(19) United States
(12) Patent Application Publication
(10) Pub. No.: US 2007/0122368 A1
(43) Pub. Date: May 31, 2007

(54) Preparation, in particular cosmetic preparation, and the production and use thereof

(75) Inventors: Azra Swistowski, Nuremberg (DE); Christina Zech, Kaufering (DE)

Correspondence Address: BACHMAN & LAPOINTE, P.C., 900 CHAPEL STREET, SUITE 1201, NEW HAVEN, CT, 06510, US

(73) Assignee: Schwan-STABILO Cosmetics GmbH & Co. KG

(21) Appl. No.: 11/641,358

(22) Filed: Dec. 18, 2006

Related U.S. Application Data
(63) Continuation-in-part of application No. 11/262,253, filed on Oct. 28, 2005.

(30) Foreign Application Priority Data
Feb. 4, 2005 (DE) 10 2005 005 486.2

(51) Int. Cl. A61K 8/31; A61K 8/37

(52) U.S. Cl. 424/70.1

(57) ABSTRACT

A preparation, in particular a cosmetic preparation, preferably in liquid or pasty form, for use on keratinic materials, in particular for decorating, coloring, improving the appearance of and caring for the skin and the skin appendages. The preparation is water-free and contains at least one wax with a dropping point above 60° C. and a liquid silicone compound.

### BACKGROUND OF THE INVENTION

The invention concerns a preparation, in particular a cosmetic preparation, preferably in liquid or pasty form, for use on the skin, on semi-mucous membranes, on mucous membranes and on keratinic materials such as hair, eyelashes and eyebrows, in particular for decorating, coloring and improving the appearance and for caring for the skin and the skin appendages. Preparations of that kind are used for example for makeup, in particular for the eyelashes and hair; such a preparation is then referred to as ‘mascara’. Basically the preparations according to the invention, with suitable adjustment and coloring, can also be used as makeup, concealer, camouflage, eyeshadow, eyeliner, lipliner, rouge, lip rouge, lip gloss, sun protection agent, sun block, temporary tattoo, colored effect sun protection for surfers and the like. Mascara preparations are known in the form of what is referred to as ‘block mascara’ or ‘cake mascara’, a solid form of preparation from which the material is removed by means of a moistened brush, in the form of ‘emulsion mascara’ in the form of O/W or W/O emulsions, or in the form of a solvent-based mascara. The use of a ‘block mascara’ is complicated if no water is available for moistening the brush or the material and another common name—namely ‘spit mascara’—shows that alternative forms of use can certainly entail serious microbiologically induced risks in terms of product and user.

Due to the type involved—at least one emulsifier must always be used in an ‘emulsion mascara’ in order to keep the continuous phase in intimate relationship with the dispersed phase. A disadvantage with such a product is generally that, after drying on the eyelashes, it can be dissolved again due to moisture from the environment, for example water, rain, perspiration and in particular the quite slightly alkaline tear fluid, whereby the durability of such preparations is reduced. The use of water-soluble polymers or polymers which can be dispersed in water also involves detrimental effects here. An example of such a polymer-bearing product is to be found in W Umbach, ‘Kosmetik’, Georg Thieme Verlag, Stuttgart, 1988, page 101. Other examples are to be found in the standard works in cosmetics literature, for example H Janistyn, ‘Handbuch der Kosmetika und Riechstoffe’, volume 3, Huthig-Verlag, 2nd edition, 1973, on pages 855-860 or G A Nowak, ‘Die kosmetischen Präparate’, Verlag Ziolkowsky, 3rd edition, 1984, pages 748-751. An emulsion mascara on a polymer base, which contains at least one wax and a pseudo-latex, should also be mentioned. To keep the emulsion stable an emulsifier must be included. Known conventional emulsifiers are often disadvantageous for use as they lead to irritation. A disadvantage with compositions of that kind is also that, after drying on eyelashes or hair, they form a brittle coating which can tear and flake off. In the case of a hair mascara, that effect can certainly be desired because such a preparation, in the form of temporary hair coloring, can later be easily removed again by brushing it out—if however fine particles flake off the eyelashes, they can land in the eye and result in mechanical irritation, or also color the region under the eye (what is referred to as the ‘panda bear effect’).

A further disadvantage with emulsion mascaras is that they are matt after application and drying. In order to particularly emphasise the eyelashes however it is advantageous if the applied layer shines.

Solvent-bearing mascara preparations are also known, which contain only little or no water at all. Polar solvents such as ethanol, propan-1-ol, propan-2-ol, acetone, methylethylketone, ethylacetate, tetrahydrofuran, dichloromethane and the like automatically rule themselves out by virtue of their odor, their severely irritating effect on skin and mucous membranes and lack of environmental compatibility. Ethanol can at best be used in amounts below 15% by volume. Non-polar isoparaffins have admittedly already been used in the past—in regard to some thereof however, a point which tells against them is the aromatics content, which excludes them for cosmetic uses and the easy flammability of the low-boiling isoparaffins, whereby specific precautions are advised in the production of preparations of that kind. If however exclusively or predominantly higher-boiling C₁₁/C₁₂ isoparaffins are chosen, preparations which adhere very well to skin and hair are admittedly obtained, which have very good transfer resistance—however their odor is not found to be pleasant.

A problem with many mascaras is that they require a special packaging. Solvent-bearing mascaras can only be packaged in those containers which are inert in relation to the solvent. If the solvent used is easily volatile or not immobilised the container must be so sealed that the solvent cannot evaporate therefrom. In addition the compatibility of the cosmetic material with the container is dependent on the respective solvent used so that different packagings have to be provided for different kinds of mascara. That is highly disadvantageous in particular for mass production.

Therefore the object of the invention was to provide a preparation in particular for coloring, improving the appearance of and caring for the skin and the skin appendages, which is shiny and which even after drying can still form a shiny layer, in order for example to produce what is referred to as the ‘wet look’. A further object of the invention was to provide a preparation which can be applied easily and uniformly to skin, semi-mucous membranes, mucous membranes, hair and eyelashes which has good coverage, which dries in a time considered by users as appropriate and as a result becomes wipe-resistant, which adheres for a long time, which does not migrate into the fine wrinkles in the skin and which is not transferred from the place of application on to other media such as for example cups, glasses, metal, textiles, or other areas of the skin and the like. That can give rise to unattractive colored impressions or films of grease, more specifically oily constituents which spread out well on the skin can migrate together with small amounts of pigment and leave unattractive traces behind on the skin. Products which do not adhere adequately to skin, semi-mucous membranes, mucous membranes, hair and eyelashes then have to be regularly re-applied.

Still a further object of the invention was to provide a preparation which can be introduced for filling purposes into all kinds of containers for cosmetic materials as it is compatible with the plastic materials used for containers of that kind. The invention further aimed to provide a preparation from which solvents cannot escape or cannot readily escape and therefore requires no special measures for packaging.

### SUMMARY OF THE INVENTION

To attain that object there is proposed a preparation as defined in the claims. The preparation according to the invention has the advantage that it is water-free and free of O/W or W/O emulsifiers and is nonetheless of a pasty consistency so that it can be easily applied to hair, eyelashes or skin. It was surprisingly found that a combination of a wax component, as is defined hereinafter, and a silicone fluid as a solubilisation agent, affords a preparation which does not suffer from the problems of the materials which are known in the state of the art, which does not crumble and which affords very durable shiny esthetic coatings.

### DETAILED DESCRIPTION

An essential constituent of the preparation according to the invention is the wax component which imparts structure to the preparation. The wax component is formed from one or more waxes and constituents which influence the viscosity and structuring of the wax or waxes. At least one of the waxes is a wax with a dropping point above 60° and, if a wax mixture is used, preferably all waxes have a dropping point above 60° C. Preferably the dropping point is above 70° C.

For the wax component the at least one wax is selected from waxes of animal, vegetable, mineral or synthetic origin or mixtures and hybrids thereof. The waxes used are of a hardness of between 2 and 40, wherein the hardness value is determined using the needle penetration method. The operation of determining the hardness value is effected in accordance with the American Standard ASTM D5: at a temperature of 25° C. a needle with a defined cone and weighing 2.5 g which is loaded with a weight of 47.5 g is caused to penetrate into a flat surface of a test body. The depth of penetration is determined in tenths of a millimeter after 5 seconds.

Beeswax, modified beeswax or so-called ‘cera bellina’, lanolin wax, Japan wax, candelilla wax, ouricuri wax, carnauba wax, rice wax, flower waxes or fruit waxes such as orange flower wax, orange wax, jasmine wax or apple wax, montan wax, microcrystalline wax, paraffin waxes, ozocerite, polyethylene waxes, waxes produced using the Fischer-Tropsch method, silicone waxes, long-chain esters such as for example cetyl palmitate, stearyl stearate, behenyl stearate, behenyl behenate, hydrated jojoba oil or mixtures thereof and their hybrids are preferably used. The amounts of such waxes or the mixtures thereof and/or their hybrids used are generally in the range of between 3 and 35% by weight, preferably between 5 and 25% by weight, and are particularly preferably between 8 and 20% by weight with respect to the finished preparation.

The wax component can further preferably include at least one non-volatile oil, at least one solid long-chain alcohol and at least one film-forming, flexible polymer or copolymer. Further ingredients such as additives and fillers, coloring agents, light protection filters which are operative in the UVA and UVB ranges, preserving agents, antioxidants, anti-settlement agents, fragrance mixtures and the like can be added, in which respect preserving agents are generally not required. The preparation according to the invention is preferably free of volatile mineral oils and triglycerides which are liquid or solid at ambient temperature, as well as conventional emulsifiers and water.

In order to make the wax component even more workable a non-volatile oil can be added. The term ‘non-volatile oil’ is used to denote a high-molecular compound based on hydrocarbon or siloxane units. Preferred non-volatile oil is high-molecular silicones such as dimethyl polysiloxane (dimethicone), phenyl trimethicone, diphenyl dimethicone and/or polybutene and polyisobutene. It is also possible to add medium-chain or long-chain carboxylic acid esters such as jojoba oil, oleyl erucate, oleyl oleate, diethyl sebacate, hexyl laurate, dibutyl adipate, isostearyl isostearate or mixtures thereof. The amount of those non-volatile oils used is generally between 1 and 30% by weight and preferably between 5 and 15% by weight.

The wax component can further also be supplemented by a long-chain alcohol with 14 to 18 and preferably 20 to 40 carbon atoms. Suitable long-chain alcohols are selected from saturated, straight-chain or branched-chain fatty alcohols which are solid at ambient temperature such as for example myristyl alcohol, cetyl alcohol, stearyl alcohol, behenyl alcohol, ceryl alcohol, myricyl alcohol or mixtures thereof. Preferably a mixture of alcohols of a chain length of between 20 and 40 carbon atoms is used. In that respect the amount of long-chain alcohols used is between 0.5 and 20% by weight, preferably between 1.5 and 15% by weight, particularly preferably between 2 and 12% by weight.

As a further constituent which can serve as a bonding agent and to improve delivery, the wax component can include a steroid-bearing material. Colophony and its derivatives, rosin derivatives as well as sterols and stanols and derivatives thereof are viewed as steroid-bearing material. In this connection uncharged derivatives are deemed to be derivatives, in particular esters with monovalent and polyvalent alcohols and copolymers; salts are excluded here. Esters which are suitable in this connection are for example the esters of rosin acid or colophony with polyvalent alcohols such as glycerine and pentaerythritol and copolymers which are made up from polyvalent alcohols, for example glyceryl units, maleic acid units and rosin acid. Sterol and sterol derivatives such as oryzanol or products which contain those compounds can also be used. Mention may be made by way of example of dipentaerythrityl hexahydroxystearate/hexastearate/hexahydrogenated rosinate, dipentaerythrityl hexahydroxy-stearate/hexastearate/hexarosinate, glyceryl dibehenate/hydrogenated rosinate, glyceryl dimyristate/hydrogenated rosinate, glyceryl hydrogenated rosinate, glyceryl rosinate, glyceryl tri-hydrogenated rosinate, glycol rosinate, methyl hydrogenated rosinate, methyl rosinate, PEG-3 dirosinate, pentaerythrityl hydrogenated rosinate, pentaerythrityl rosinate, TEA-ronsinate, triethylene glycol hydrogenated rosinate, and/or triethylene glycol rosinate; or mixtures thereof. Vegetable extracts which contain sterol-like compounds, for example rice bran wax are also suitable. The steroid derivative is incorporated in such amounts that it achieves the desired effect. Preferably an amount of 0.1 to 10% by weight, particularly preferably 0.5 to 4% by weight, is used. The compounds can also contribute further advantageous properties, for example promote film formation and reinforce the pleasant feel on the skin.

In order to plasticise the wax component or make it pasty, a silicone oil is used as a solubilisation agent, which can be a straight, branched or cyclic silicone with between 1 and 20 siloxane units, preferably between 3 and 8 siloxane units. Particularly preferably the silicone oil is selected from straight short-chain dimethyl siloxanes with between 2 and 8 and preferably 2 and 6 siloxane units such as for example hexamethyl disiloxane, octamethyl trisiloxane, decamethyl tetrasiloxane and/or dodecamethyl pentasiloxane, or cyclic silicone oils with between 4 and 7 siloxane units such as for example octamethyl cyclotetrasiloxane, decamethyl cyclopentasiloxane, dodecamethyl cyclohexasiloxane or mixtures thereof. The amount of those low-molecular silicone oils used is in the range of between 10 and 80% by weight, preferably between 15 and 65% by weight and quite particularly preferably between 25 and 50% by weight.

The preparation according to the invention is thus a combination of a wax component and a silicone fluid. In order to process those essential constituents to afford a homogeneous composition, a preferred embodiment involves the addition of a compatibilisation agent. The compatibilisation agent is a compound which mediates between the hydrophobic wax compound and the lipophobic and hydrophobic silicone fluid. Alkyl polysiloxanes, in particular dimethylsiloxanes which carry long-chain alkyl residues as terminal groups and/or side chains are suitable for that purpose. Preferred alkyl polysiloxanes are selected from alkyl dimethicones which can carry alkyl residues of differing chain length. The alkyl residues can have between 10 and 36 carbon atoms. In addition they can also have hydroxyl groups and/or be connected by way of ether bridges to shorter alkyl chains. Esters of dimethiconols are to be ascribed to alkyl dimethicones in the broadest sense. Mentioned here by way of example are substances such as lauryl dimethicone, cetyl dimethicone, cetearyl dimethicone, behenyl dimethicone, C24-28 alkyl dimethicone, bishydroxyethoxy propyl dimethicone, phenyl propyl silsesquisiloxane, dimethiconol stearate, hydroxy propyl dimethicone behenate and mixtures thereof. In that respect the amount used is between 0.5 and 35% by weight, preferably between 1.5 and 25% by weight, quite particularly preferably between 3 and 12% by weight.

After application the preparation according to the invention is to remain properly at the place of application and not move away therefrom. In order to improve adhesiveness a non-volatile oil or a carboxylic acid ester can be added to the preparation according to the invention. Examples of suitable non-volatile silicone oils are dimethyl polysiloxanes with a molecular weight of between 50,000 and 1,000,000, preferably between 200,000 and 800,000 and particularly preferably between 300,000 and 500,000, which can be substituted with phenyl groups. It is possible to use for example dimethicone, phenyl trimethicone or diphenyl dimethicone with a molecular weight in the specified range. Examples of carboxylic acid esters which are suitable as a bonding agent are jojoba oil, oleyl erucate, oleyl oleate, diethyl sebacate, hexyl laurate, dibutyl adipate, isostearyl isostearate or mixtures thereof. The amount of those non-volatile oils used is generally between 1 and 30% by weight and preferably between 7 and 25% by weight.

To improve applicability, durability and transfer resistance, preferably a film-forming, flexible polymer or copolymer is used, having a dropping point of at least 30° C. Preferably the polymer or copolymer is selected from olefin maleic acid copolymers, while particularly preferably a long-chain alkyl/isoalkyl maleate MA terpolymer is used. C30-38 olefin/isopropyl maleate/MA copolymer, cetearyl dimethicone/vinyl dimethicone cross polymer, octadecene/MA copolymer, methoxy aminodimethicone silsesquioxane copolymer or mixtures thereof have proven to be particularly suitable. Thereof, between 0.1 and 20% by weight, preferably between 0.5 and 12% by weight and quite particularly preferably between 2 and 8% by weight is used.

The preparation according to the invention can also contain further ingredients as are usual for cosmetic compositions. Those ingredients serve to adjust properties such as viscosity, structure, flowability and consistence and the adhesiveness of the application as well as color and esthetic parameters.

In a preferred embodiment the preparation according to the invention, for adjusting consistency, includes a silicon dioxide or silica, in particular highly dispersed silicic acid with a large surface area which can absorb the silicone fluid and thereby contributes to compatibilisation and the avoidance of phase separation. sulfide when using ultramarine blue or the possible liberation of hydrogen when using metal powders is also not to be expected because of the absent water as a reaction partner. When using silica, surface-coated, preferably hydrophobised products are advantageously used.

In sun protection agents, in particular so-called ‘sun blocks’, besides oil-soluble light filter substances such as for example isoamyl or ethylhexyl-p-methoxy cinnamate or 4-methylbenzylidene camphor, it is also possible to use combinations with metal oxides such as for example titanium dioxide, cerium oxide, zinc oxide and zirconium oxide in the form of so-called ‘nanopigments’, of mean particle sizes in the range of between 5 and 25 nm.

For the purposes of viscosity regulation and stabilisation of the preparation according to the invention, it is certainly also possible to use per se known additives such as bentonites, hectorites, montmorillonites or also organically modified derivatives of those additives and their combinations, which then possibly have to be activated in a suitable manner known to the man skilled in the relevant art. As is known, small additions of diethyl carbonate, propylene carbonate or alcohol solutions of α-hydroxy acids are suitable for that purpose. Naturally it is also possible to produce oleogels using 12-hydroxystearic acid and behenates, for example behenamidopropyl dimethylamine behenate—the corresponding processes are known to the man skilled in the art for example from DE 102 01 370 C2.

As the preparations according to the invention are water-free, it is generally also possible to dispense with the use of preserving agents. If antioxidants are used, those which are usually employed are suitable. Preferably cosmetically acceptable products such as vitamin E acetate are used.

The preparation may also contain fillers such as for example talcum, kaolin, healing earth, smectite, starch and modified starch, non-swelling starches, cellulose, polytetrafluoroethylene powder (Teflon), polyamide powder (Nylon), mica, insoluble metal soaps such as Mg stearate, Ca stearate, Sr stearate or Zn stearate and/or inorganic or organic pigments and lakes of organic coloring agents. The following may be mentioned by way of example of the latter: titanium dioxide, zinc oxide, yellow, red or black iron oxide, chromium oxide green, chromium hydroxide green, ultramarine, Berlin Blue (Ferric Blue), manganese violet, carmine, pearlescent pigments, for example micas coated with titanium dioxide and colored micas coated with titanium dioxide and metal oxides, bismuth oxychloride, bismuth oxychloride coated with metal oxides, flake-form metal powders of aluminum, bronze, brass, copper, silver and gold, as well as lakes of organic dyes with aluminum, barium, calcium, strontium or zirconium or mixtures of the specified substances. That list is only by way of example and is in no way definitive. The amounts used in that respect are within the usual limits and are preferably in accordance with the maximum amounts regulated by the respective cosmetic legislation. The quantitative proportions of fillers and coloring agents are in that connection in a range of between 0.01 and 70% by weight, preferably in a range of between 1 and 55% by weight and quite particularly preferably in a range of between 2 and 25% by weight.

The high level of sheen which can be achieved and the high level of color intensity, caused by the refractive index of the silicone used, is advantageous when employing the coloring agents set forth hereinbefore by way of example, in water-free preparations. The preparations are independent of any influences caused by pH-values, for example the development of hydrogen

The preparations according to the invention are present in viscous form or in the form of workable pastes and can be easily and uniformly distributed on the skin, on semi-mucous membranes, on mucous membranes and on hair and eyelashes. The viscosity of the preparations according to the invention is adjusted according to the desired purpose of use and is in the range of between 50 and 1500 mPas in the case of the liquid to viscous preparations and in the range of between 1.5 and 500 Pas in the case of the pasty preparations. In that respect the viscosity is determined in conventional manner with a commercially available viscosimeter at a rotary speed of 1 rpm with a plate/ball or plate/plate measuring device, in the case of the latter with a gap width of 0.4 mm. After drying, the preparations according to the invention form at the place where they are applied an elastic layer which is perceived as being pleasant and which does not move, which does not run, which does not migrate and which can remain at the place at which it is applied for the entire day. It can be removed again in a manner which is known to the users—by suitable makeup removal agents or cloths, by means of a cleaning lotion or by washing off with fine soap or with corresponding mild tenside preparations. An advantage of the preparations according to the invention is that they can be filled into any kind of container as they are compatible with the usual polymers employed for containers of that kind, that is to say they do not react therewith and they are not altered thereby. As the preparations do not contain any solvents which are given off upon storage under normal conditions, there is no need for special measures to prevent solvent from escaping. The preparations according to the invention can therefore be filled in known manner into containers suitable for the desired purpose of use such as bottles with applicators contained therein (spatula, fine brush, coarse brush, and the like), jars, tubes, pots or tubs (bowls) and can be taken therefrom again by the user. However, because of the improved hygiene conditions that this involves, they can also be introduced into suitable applicator devices, referred to as ‘dispenser mechanisms’, and applied therefrom. So-called pressure-actuated metering dispensers with a trailing piston which subsequently moves along with the material are basically known. The possibility of filling a preparation in a container in the form of a dual-chamber aerosol is also known. Applicator devices as are known for example from U.S. Pat. No. 6,238,117 or U.S. Pat. No. 6,309,128 are suitable for the application of small amounts as are required for example in the lip and eye region as they allow very attractive fine metering.

If the preparations according to the invention are produced as ‘mascara preparations’ for coloring the eyelashes, then they are intended not only to impart color to the eyelashes but also to strengthen them, in order to cause them to appear visually thicker and stronger and to give them curl. The preparations are then usually colored black, brown, blue and dark grey. If the intention is only to make the natural color of eyelashes stronger and more impressive, the recommendation is for the mascara preparation to be produced in transparent form without fillers and coloring agents. If the intention is to achieve particular color effects under long-wave UV-light (so-called ‘black light’), in order for example to achieve particular effects under disco lighting, it is possible to add to such a mascara preparation titanium dioxide in the form of nanopigments or stilbene derivatives which are allowed for cosmetics use. The current fashion is for the tips of the eyelashes to be accentuated with contrasting colors, for example light green, moss-green, yellow, orange or red. Preferably the above-mentioned inorganic pigments which are allowed for the region of the eyes are suitable for that purpose, as coloring agents, but also for example lakes of organic dyes such as FD&C Blue No 1 (C.I.-No 42090), FD&C Red No 40 (C.I.-No 16035), or FD&C Yellow No 50 (C.I.-No 19140). If the preparation according to the invention is to be used as hair mascara, the color selection is naturally more versatile as in that case then it is also possible to use lakes of organic dyes which are only allowed for external use. If preparations are wanted which give particular color effects under disco lighting, it is possible to use the fluorescent dyes which are suitable for that purpose and which are sufficiently known to the man skilled in the relevant art—see in that respect for example WO 2004/108108. It is also possible to use fine glitter particles of material-colored polymer carriers such as polyesters, polycarbonates or the like, in effect preparations. Mascara preparations which contain fine fibers, for example Nylon, silk, cellulose, polyester, Teflon, Rayon and the like have already long been usual for strengthening and lengthening eyelashes.

Containers for mascara are commercially available and are sufficiently known to the man skilled in the art—he only has to consider here that the selected brush provides for good wetting of the eyelashes and in so doing separates them and that the brush and the scraper portion are well matched to each other. If a mascara preparation and an effect mascara are offered together, then a packaging which can be combined from two individual units is recommended—even those combination packagings in which one unit can be provided with a smaller volume and a smaller brush have in the meantime become commercially available. As normally a person has more hair on the head than eyelashes, a larger container naturally has to be adopted for hair mascara.

The preparation according to the invention will now be described in detail by means of the following Examples which however do not definitively describe it. In that respect the amounts are given in percent by weight, in each case with respect to the total weight of the respective preparation. In an individual case the amounts involved can be slightly greater than or less than the specified amounts, in which case nonetheless the preparations according to the invention are produced. That is in no way unexpected to the man skilled in the relevant art, in consideration of the widely available great variety of suitable components of such preparations, so that the man skilled in the art knows that, when the amounts used exceed or are less than the specified amounts in that way, this does not constitute a departure from the range of the present invention. The INCI names which are known to the man skilled in the relevant art are used to identify the raw materials involved.

### EXAMPLE 1

### Pasty Mascara Preparation

| | |
|---|---|
| Octamethyl trisiloxane | 35.900 |
| Dimethicone (mw 500,000) | 17.500 |
| Cera microcristallina (81-84° C.) | 12.000 |
| C24-28 alkyldimethicone | 8.000 |
| Candelilla cera | 7.000 |
| C30-38 olefin/isopropyl maleate/MA copolymer | 6.500 |
| C20-C40 alcohols | 2.500 |
| Black iron oxide (C.I. No 77.499) | 8.000 |
| Silica | 2.000 |
| Tocopherol acetate | 0.350 |
| Fragrance | 0.250 |

For production purposes the raw materials of items Nos 2-6 are melted at about 80° C., then the coloring agent (black iron oxide) and the silica are added and the mixture is then passed three times through a three-roll mill. The mixture is now transferred into a vacuum process installation, octamethyl trisiloxane, tocopherol acetate and the fragrance mixture are added thereto, the mixture is thoroughly mixed, deaerated under a slight vacuum, cooled to about 28-30° C. and transferred into storage containers.

For the production of further colors, instead of just the black iron oxide, mixtures of black, red and yellow iron oxide are used to produce a brown mascara preparation, mixtures of black iron oxide with titanium dioxide are used to produce a gray mascara preparation and ultramarine, possibly also using titanium dioxide, is used for the production of a blue mascara preparation. Effect colors can be produced by the use of yellow and/or red iron oxide (yellow, orange, red mascara preparation), chrome oxide green (moss-green mascara preparation), chromium hydroxide green (turquoise mascara preparation), possibly also in each case mixed with titanium dioxide. In that respect it is a matter for the discretion of the man skilled in the art to effect variations in the foregoing Example, according to the desired requirements in terms of color shade and color intensity. In that respect reductions in the amount of pigment can be compensated by an increase in the proportion of volatile oils or by the addition of uncoated mica or talcum.

### EXAMPLE 2

### Pasty, Colorless Mascara Preparation

| | |
|---|---|
| Decamethyl tetrasiloxane | 41.600 |
| Dimethicone (mw 350,000) | 12.500 |
| Cera microcristallina (81-84° C.) | 12.000 |
| C24-28 alkyldimethicone | 5.500 |
| Carnauba wax (Copernicia Cerifera) | 6.500 |
| C30-38 olefin/isopropyl maleate/MA copolymer | 4.500 |
| C20-C40 alcohols | 4.500 |
| Titanium dioxide (nanopigment 10-20 nm) | 8.000 |
| Silica | 2.500 |
| Silica silylate | 1.800 |
| Tocopherol acetate | 0.350 |
| Fragrance | 0.250 |

The production of Example 2 is effected in a similar manner to Example 1. This mascara preparation produces on the eyelash an almost colorless and transparent coating which thickens and slightly lengthens the eyelash. Under long-wave UV-light that preparation exhibits an intensive, light-blue lighting effect. In order to obtain a product which is entirely colorless even under UV-light, the titanium dioxide nanopigment is replaced by the corresponding amount of decamethyl tetrasiloxane.

### EXAMPLE 3

### Pasty Hair Mascara

| | |
|---|---|
| Decamethyl tetrasiloxane | 37.900 |
| Dimethicone (mw 500,000) | 14.500 |
| Cera microcristallina (81-84° C.) | 12.000 |
| C24 28 alkyldimethicone | 5.500 |
| Beeswax, white | 6.500 |
| C30-38 olefin/isopropyl maleate/MA copolyrner | 4.500 |
| C20-C40 alcohols | 4.500 |
| Titanium dioxide | 2.500 |
| Red iron oxide (C.I.-No 77.491) | 5.000 |
| Yellow iron oxide (C.I.-No 77.492) | 1.500 |
| Polyester-3, Red 28 *) | 5.000 |
| Tocopherol acetate | 0.350 |
| Fragrance | 0.250 |

The production of Example 3 is similar to Example 1. That mascara preparation is particularly suitable for the temporary coloring of individual strands and produces on the hair an orange-yellow coloring which under long-wave UV-light exhibits a bright red fluorescence.

### EXAMPLE 4

### Pasty, Effect Sun Protection for Surfers

| | |
|---|---|
| Octamethyl trisiloxane | 40.900 |
| Dimethicorie (mw 500,000) 8.500 | |
| Cera microcristallina (81-84° C.) | 5.000 |
| C24-28 alkyldimethicone | 5.500 |
| Beeswax, white | 2.500 |
| C30-38 olefin/isopropyl maleate/MA copolymer | 4.500 |
| C20-C40 alcohols | 3.500 |
| Isoamyl p-methoxycinnamate | 4.000 |
| 4-Methylbenzylidene camphor | 3.500 |
| Buxus chinensis (jojoba oil) | 7.000 |
| Titanium dioxide (nanopigment 10-20 nm) | 8.000 |
| Titanium dioxide (C.I.-No 77.891) | 1.500 |
| Ultramarine (C.I.-No 77.007) | 5.000 |
| Tocopherol acetate | 0.350 |
| Fragrance | 0.250 |

The production of Example 4 is effected similarly to Example 1. The result obtained is a radiantly blue paste with the effect of a sun block. If color variants are desired, the ultramarine can also be replaced by yellow or red iron oxide, chromium hydroxide green, chromium oxide green, manganese violet, titanium dioxide or mixtures thereof. Applied by means of a roll-on applicator, it is possible to apply lines or drawings to the skin.

### EXAMPLE 5

### Pasty, Colorless Sun Block

| | |
|---|---|
| Octamethyl trisiloxane | 41.400 |
| Dimethicone (mw 500,000) | 9.500 |
| Cera microcristallina (81-84° C.) | 7.000 |
| C24-28 alkyldimethicone | 6.500 |
| Ouricuri wax | 4.500 |
| C30-38 olefin/isopropyl maleate/MA copolymer | 4.500 |
| C20-C40 alcohols | 3.500 |
| Isoamyl p-methoxycinnamate | 5.500 |
| 4-Methylbenzylidene camphor | 4.500 |
| Buxus chinensis (jojoba oil) | 3.000 |
| Titanium dioxide (nanopigrnent 10-20 nm) | 8.000 |
| Quaternium-is hectorite | 1.500 |
| Tocopherol acetate | 0.350 |
| Fragrance | 0.250 |

The production of Example 5 is effected similarly to Example 1. The result obtained is a colorless translucent paste which is suitable for filling in tubes. It affords a high level of light protection in the region around SF 25-30.

### EXAMPLE 6

### Pasty Eyeshadow

| | |
|---|---|
| Octamethyl cyclotetrasiloxane | 35.000 |
| Decamethyl cyclopentasiloxane | 14.000 |
| Dimethicone (mw 500,000) | 3.600 |
| Ouricuri wax | 6.200 |
| C20-C40 alcohols | 4.800 |
| C30-38 olefin/isopropyl maleate/MA copolymer | 5.200 |
| Hydroxypropyl dimethicone behenate | 7.200 |
| Lauryl dimethicone | 7.200 |
| Silica | 2.000 |
| Titanated mica (C.I.-No 77891) | 8.000 |
| Mica | 2.000 |
| Chromium hydroxide green (C.I.-No 77289) | 3.200 |
| Ultramarine (C.I.-No 77007) | 0.800 |
| Tocopherol | 0.600 |
| Fragrance | 0.200 |

The production of Example 6 is effected similarly to Example 1, but the pearl pigments are added only after the second pass through the three-roll mill. The powerfully turquoise material with a bright pearl sheen is suitable for filling into small pots, or for improved handling and product hygiene, an elongate container with an applicator fixed to a stem, a so-called ‘deer foot applicator’, as a holder, which is connected to the closure cap.

### EXAMPLE 7

### Fluid Foundation, Water-Resistant

| | |
|---|---|
| Octamethyl cyclotetrasiloxane | 34.200 |
| Decamethyl cyclopentasiloxane | 42.700 |
| Dimethiconol stearate | 0.850 |
| Bishydroxyethoxypropyl dimethicone | 5.100 |
| Cetearyl dimethicone/vinyldimethicone cross-polymer | 1.700 |
| Cera microcristallina (81-84° C.) | 2.200 |
| Candelilla cera | 0.900 |
| C20-28 alkyl dimethicone | 1.700 |
| C20-C40 alcohols | 1.700 |
| C30-38 olefin/isopropyl maleate/MA copolymer | 1.700 |
| Tocopherol | 0.550 |
| Silica | 0.450 |
| Titanium dioxide (C.I.-No 77891) | 3.500 |
| Yellow iron oxide (C.I.-No 77492) | 1.200 |
| Red iron oxide (C.I.-No 77491) | 0.850 |
| Black iron oxide (C.I.-No 77499) | 0.450 |
| Fragrance | 0.250 |

The production of Example 7 is effected similarly to Example 1. The result obtained is a light-brown pasty preparation which has good coverage and is long-lasting, with good transfer resistance and which is highly resistant to moisture, for example perspiration. It is highly suitable for being filled-into tubes.

### EXAMPLE 8

### Fluid Lipliner

| Octamethyl cyclotetrasiloxane 68.000 | |
|---|---|
| Cera microcristallina | 6.800 |
| Candelilla cera | 1.400 |
| Dimethicone (mw 500,000) | 4.100 |
| C24-28 alkyl dimethicone | 2.700 |
| C20-C40 alcohols | 0.700 |
| Tocopherol | 0.500 |
| Octadecene/MA copolymer | 0.700 |
| Diethyl sebacate | 0.900 |
| Silica | 0.400 |
| Titanium dioxide (C.I.-No 77891) | 4.100 |
| Red iron oxide (C.I.-No 77491) | 6.000 |
| FD&C Red No 40 (C.I.-No 16035) | 3.500 |
| Fragrance | 0.200 |

The production of Example 8 is effected similarly to Example 1, in which respect, in order to obtain a material which can be processed on a three-roll mill, about 20-25% of the volatile silicone oil is added to the hot material. The result obtained is a powerfully red, still fluid preparation which affords good coverage and which can be applied in small containers by means of a fiber tip fitted into the closure cap. The preparation is transfer-resistant and does not migrate into the fine wrinkles of the skin.

### EXAMPLE 9

### Fluid Eyeliner

| | |
|---|---|
| Octamethyl trisiloxane | 52.000 |
| Octamethyl cyclotetrasiloxane | 20.000 |
| Cera microcristallina | 5.000 |
| Rice bran wax | 2.000 |
| C20-40 alcohols | 1.600 |
| Dimethicone (mw 500,000) | 2.100 |
| C30-38 olefin/isopropyl maleate/MA copolymer) | 1.800 |
| Phenylpropylpolysilsesquisiloxane | 2.400 |
| Tocopherol | 0.400 |
| Silica | 0.500 |
| Black iron oxide (C.I.-No 77499) | 10.000 |
| Ultramarine (C.I.-No 77007) | 1.300 |
| Carmine (C.I.-No 75470) | 0.700 |
| Fragrance | 0.200 |

The production of Example 9 is effected similarly to Example 8. The result obtained is a fluid preparation of a deep black color and of good durability and transfer resistance. For ease of handling, it is filled into the containers referred to in Example 8.

### EXAMPLE 10

### Pasty Covering Cream for Couperose, Water-Resistant

| | |
|---|---|
| Octamethyl trisiloxane | 9.200 |
| Octamethyl cyclotetrasiloxane | 46.000 |
| Decamethyl cyclopentasiloxane | 15.200 |
| Cera microcristallina | 3.800 |
| C24-28 alkyldimethicone | 5.700 |
| C20-40 alcohols | 1.900 |
| C30-38 olefin/isopropyl maleate/MA copolymer | 1.900 |
| Methoxy amodimethicone silsesquioxane copolymer | 1.900 |
| Dimethicone (mw 500,000) | 4.700 |
| Tocopherol | 0.450 |
| Mica | 1.000 |
| Titanium dioxide (C.I.-No 77891) | 4.500 |
| Chromium hydroxide green (C.I.-No 77269) | 3.500 |
| Fragrance | 0.250 |

The production of Example 10 is effected similarly to Example 1. The result obtained is a light-turquoise preparation with good coverage and very good transfer resistance, which can be easily distributed uniformly on the skin. The preparation is suitable for filling in tubes. It serves to conceal skin anomalies such as pigment spots and small visible blood vessels (couperose and rosacea), by virtue of its complementary color. As the preparation is water-resistant and is not dissolved by W/O emulsions, after drying on the skin, a suitable makeup can be applied according to the taste of the respective wearer.

1. A water-free cosmetic preparation in liquid or pasty form, comprises a wax component and a liquid silicone compound as a solubilisation agent, wherein the wax component is formed from at least one wax with a dropping point above 60° C., at least one non-volatile oil and/or a long-chain alcohol and/or compatibilisation agent and/or a film-forming polymer or copolymer with a dropping point of at least 30° C.

2. A preparation as set forth in claim 24, wherein the preparation is free of non-volatile mineral oils and triglycerides which are liquid or solid at ambient temperature.

3. A preparation as set forth in claim 2, wherein the wax has a needle penetration in accordance with ASTM D5 of between 2 and 40, determined at a temperature of 25° C. and with a penetration time of 5 sec, with a total weight of the test body of 50 g.

4. A preparation as set forth in claim 1, wherein the wax is selected from the group consisting of beeswax, modified beeswax, candelilla wax, carnauba wax, ouricuri wax, rice wax, montan wax, microcrystalline wax, paraffin wax, ozocerite, polyethylene wax, wax produced in accordance with the Fischer-Tropsch process, silicone wax and mixtures thereof.

5. A preparation as set forth in claim 1, further comprising a further wax and/or long-chain esters which are selected from the group consisting of lanolin wax, Japan wax, orange flower wax, orange wax, cetyl palmitate, stearyl stearate, behenyl stearate, behenyl behenate, hydrated jojoba oil and mixtures thereof.

6. A preparation as set forth in claim 5, wherein the amount of the waxes used is in the range of between 3 and 35% by weight.

7. A preparation as set forth in claim 5, wherein the amount of the waxes used is in the range of between 5 and 25% by weight

8. A preparation as set forth in claim 5, wherein the amount of the waxes used is in the range of between 8 and 20% by weight.

9. A preparation as set forth in claim 1, wherein the liquid silicone compound is selected from linear, branched or cyclic silicone oils and mixtures thereof.

10. A preparation as set forth in claim 9, wherein the silicone oil is selected from the group consisting of hexamethyl disiloxane, octamethyl trisiloxane, decamethyl tetrasiloxane, dodecamethyl pentasiloxane, octamethyl cyclotetrasiloxane, decamethyl cyclopentasiloxane, dodecamethyl cyclohexasiloxane and mixtures thereof.

11. A preparation as set forth in claim 9, wherein the amount of the liquid silicone compound used is between 10 and 80% by weight.

12. A preparation as set forth in claim 9, wherein the amount of the liquid silicone compound used is between 15 and 65% by weight.

13. A preparation as set forth in claim 9, wherein the amount of the liquid silicone compound used is between 25 and 50% by weight.

14. A preparation as set forth in claim 1, wherein the wax component includes a non-volatile oil selected from high-molecular compounds based on hydrocarbon or siloxane units, medium-chain and long-chain carboxylic acid esters and mixtures thereof.

15. A preparation as set forth in claim 14, wherein the non-volatile oil is selected from high-molecular dimethyl polysiloxane (dimethicone), phenyl trimethicone, diphenyl dimethicone, and mixtures thereof.

16. A preparation as set forth in claim 15, wherein the dimethyl polysiloxane is selected from the long-chain linear dimethyl polysiloxanes of a molecular weight above 100,000 Daltons.

17. A preparation as set forth in claim 15, wherein the dimethyl polysiloxane is selected from the long-chain linear dimethyl polysiloxanes of a molecular weight above 250,000 Daltons.

18. A preparation as set forth in claim 15, wherein the dimethyl polysiloxane is selected from the long-chain linear dimethyl polysiloxanes of a molecular weight above 450,000 Daltons.

19. A preparation as set forth in claim 14, wherein the medium-chain and long-chain carboxylic acid esters are selected from the group consisting of jojoba oil, oleyl erucate, oleyl oleate, hexyl laurate, dibutyl adipate, diethyl sebacate, isostearyl isostearate and mixtures thereof.

20. A preparation as set forth in claim 14, wherein the amount of the non-volatile oils is between 1 and 30% by weight.

21. A preparation as set forth in claim 14, wherein the amount of the non-volatile oils is between 2 and 25% by weight.

22. A preparation as set forth in claim 1, wherein the preparation contains at least one alkyl dimethicone selected from the group consisting of lauryl dimethicone, cetyl dimethicone, cetearyl dimethicone, behenyl dimethicone, C24-28 alkyl dimethicone, bishydroxyethoxy propyl dimethicone, phenyl propyl silsesquisiloxane, dimethiconol stearate, hydroxypropyl dimethicone behenate, and mixtures thereof.

23. A preparation as set forth in claim 22, wherein the amount of alkyl dimethicone used is between 0.5 and 35% by weight.

24. A preparation as set forth in claim 22, wherein the amount of alkyl dimethicone used is between 1.5 and 25% by weight.

25. A preparation as set forth in claim 22, wherein the amount of alkyl dimethicone used is between 3 and 12% by weight.

26. A preparation as set forth in claim 24, further comprising at least one long-chain alcohol which is solid at ambient temperature and selected from the group consisting of saturated straight-chain or branched-chain alcohols of a chain length of between 14 and 48 carbon atoms and mixtures thereof.

27. A preparation as set forth in claim 26, wherein the long-chain alcohol which is solid at ambient temperature is selected from the group consisting of myristyl alcohol, cetyl alcohol, stearyl alcohol, behenyl alcohol, ceryl alcohol, myricyl alcohol, and mixtures thereof.

28. A preparation as set forth in claim 26, wherein the amount of the alcohols used is between 0.5 and 20% by weight.

29. A preparation as set forth in claim 26, wherein the amount of the alcohols used is between 2.5 and 15% by weight.

30. A preparation as set forth in claim 26, wherein the amount of the alcohols used is between 4 and 12% by weight.

31. A preparation as set forth in claim 1, further including a film-forming, flexible polymer or copolymer having a dropping point of at least 30° C.

32. A preparation as set forth in claim 31, wherein the film-forming, flexible polymer or copolymer is selected from the group consisting of C30-38 olefin/isopropyl maleate/MA copolymer, cetearyl dimethicone/vinyl dimethicone cross-polymer, octadecene/MA copolymer, methoxy amodimethicone silsesquioxane copolymer, and mixtures thereof.

33. A preparation as set forth in claim 32, wherein the amount of polymer or copolymer used is between 0.1 and 20% by weight.

34. A preparation as set forth in claim 32, wherein the amount of polymer or copolymer used is between 0.5 and 12% by weight.

35. A preparation as set forth in claim 32, wherein the amount of polymer or copolymer used is between 2 and 8% by weight.

36. A preparation as set forth in claim 1, wherein the preparation is in the form of makeup, concealer, camouflage, eyeshadow, eyeliner, lipliner, rouge, lip rouge, lip gloss, sun protection agent, sun block, temporary tattoo, mascara, hair mascara, effect sun protection for surfers and as covering cream in relation to couperose and rosacea.
